(19) United States
(12) Patent Application Publication
(10) Pub. No.: US 2005/0074804 A1
(43) Pub. Date: Apr. 7, 2005

(54) Amplification of polynucleotide sequences by rolling circle amplification

(76) Inventors: Youxiang Wang, Palo Alto, CA (US); Yaping Zong, San Jose, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(21) Appl. No.: 10/952,020

(22) Filed: Sep. 27, 2004

Related U.S. Application Data
(60) Provisional application No. 60/506,218, filed on Sep. 26, 2003.

(51) Int. Cl. C12Q 1/68

(52) U.S. Cl. 435/6

(57) ABSTRACT

The present invention is directed to methods of amplification and detection of nucleic acids by rolling circle amplification. The methods of the present invention may be used to amplify nucleic acids for detection and cloning. The methods are particularly suited to RNA.

### CROSS-REFERENCE TO RELATED APPLICATION

This application claims priority to U.S. Ser. No. 60/506,218, filed Sep. 26, 2003, entitled Amplification of Polynucleotide Sequences by Rolling Circle Amplification by inventors Youxiang Wang and Yaping Zong.

### FIELD OF THE INVENTION

The present invention is in the field of methods of amplification of nucleic acids by rolling circle amplification.

### BACKGROUND

Cloning and detection of nucleic acids, particularly RNAs, is routinely performed in molecular biology today. Often detection or cloning of nucleic acids is performed on complex mixtures of nucleic acids, where the particular nucleic acid of interest is under-represented. In such situations, the nucleic acid of interest is usually amplified prior to cloning or detection.

Various nucleic acid amplification methods have been invented in recent years, including methods based on cycling temperature such as PCR, LCR, and SPA, and methods using isothermal amplification such as NASBA, RCA, TMA, Q beta replicase and SDA. Isothermal amplification methods simplify the instrumentation and make integrated automation easier.

PCR is widely used for amplification of nucleic acids, though it can produce less than optimal results. For example, artifacts can arise due to mis-priming or mis-hybridization of primer oligonucleotides. In addition, cloning longer nucleic acids may yield substantial by-products in the form of less than full length clones of the nucleic acid of interest. In addition, PCR requires specialized precision temperature cycling equipment that increases the cost.

Currently, mRNA amplification methods require synthesis of the first strand of cDNA and then the second strand of cDNA by taking advantage of the polyA tail. The resulting double strand cDNA may contain a promoter site for the DNA-dependent RNA polymerase to produce single strand RNA products, which are antisense to the initial target. See, for example, U.S. Pat. No. 5,716,785. The whole process is tedious and the resulting products are mostly 3′ biased. Furthermore, two primers are usually required in these methods in order to synthesize double strand cDNA. On the one hand, two primers offer an advantage in amplifying a target nucleic acid from complex mixtures. On the other hand, two primers impair the ability to selectively amplify a specific strand of a nucleic acid target.

PCR-based amplification methods are commonly used in detection and quantification of nucleic acids. One widely used gene quantification method is TaqMan RT-PCR. Besides inheriting the drawbacks of PCR, it is very expensive and very difficult to apply to detection of multiple genes in a single pot reaction.

Rolling circle amplification (RCA) has recently been developed as an alternative method of amplification to PCR. RCA takes advantage of the topology of a circular nucleic acid as an endless circular line. Once an appropriate polymerase has initiated replication or transcription of the circular nucleic acid, the polymerase will continue until it falls off or is otherwise removed. Rolling circle amplification is isothermal, thus eliminating the need to use a thermo-stable polymerase or a temperature cycling apparatus. The thermal cycling process takes time with each cycle requiring heating blocks to change and transmit a temperature change and increases the expense of the method due to the requirement for a thermal cycling apparatus.

Various detection and amplification methods have been developed utilizing RCA. In U.S. Pat. No. 5,871,921, Landegren et al. describe a method in which rolling circle amplification may be used for detection of genomic variants. In this assay, a detectable nucleic acid probe is hybridized to a single stranded nucleic acid target. The probe will hybridize with the target nucleic acid only if the targeted sequence is present. The hybridized probe ends are then covalently connected to form a continuous loop of probe nucleic acid. Following the formation of the continuous loop, the probe/target is subjected to conditions that would remove probes that did not form a continuous circuit, such as denaturing the probe/target hybrid or subjecting the probe to exonuclease activity to remove the non-cyclized probes. The target molecule may then be detected by determination of the presence of the interlocking catenated probe. Analysis of the reaction product requires separation of target DNA that does not have a tethered ligated probe from target DNA that does have the tethered ligated probe.

An alternative method of using the rolling circle amplification process is disclosed in U.S. Pat. No. 5,648,245 to Fire et al. The reference describes a four-step process for generating a concatamer library. In the procedure, the first step is to generate an amplification target circle by annealing ends of a padlock probe to a target nucleic acid sequence followed by ligation of the ends of the padlock probe to form a continuous loop. Once the amplification target circle is formed, the second step is to create a single stranded tandem-sequence DNA by rolling circle amplification of the amplification target circle. The third step requires converting the single stranded tandem-sequence DNA to double stranded tandem-sequence DNA. Finally, the double stranded tandem-sequence DNA is cloned or used for in vitro selection.

U.S. Pat. No. 5,866,377 to Schon uses rolling circle amplification as a method to detect variants in a nucleic acid sequence. In this method, a padlock probe hybridizes to a single stranded nucleic acid such that the ends are adjacent to each other. A ligase then joins the ends of the probe. The ligation reaction will be carried out only if the target nucleic acid contains a specific variant base at the locus near the end base of one of the probe ends. Detection of the presence of the catenated probe on the target nucleic acid indicates the presence of the specific variant. U.S. Pat. No. 5,854,033 to Lizardi describes a similar assay where the catenated probe is used to produce tandem-sequence DNA by rolling circle amplification. The tandem sequence is detected to determine the amount of target sequence present.

U.S. Pat. No. 6,287,824 describes inserting double strand cDNA into a vector and then amplifying the vector with rolling circle amplification. Generating circular vectors by this method is time consuming, and the resultant RCA products contain a majority of unrelated or useless sequence from the vectors. The efficiency of the RCA amplification is low due to the size of the circular vectors.

U.S. Pat. No. 6,323,009 describes amplifying circular genomic DNA present in colony and plague by using random sequence oligonucleotide primers. It can only, however, amplify the circular genomic DNA already present in the medium, not the linear double genomic DNA.

Current RCA detection methods are complicated and have numerous drawbacks. A) They require using a padlock probe to hybridize to the target to create a circle. The efficiency of using a padlock probe to form a circle is low, especially if RNA is a template. It is even more complicated since the ligation efficiency is sequence dependent. It is difficult to quantify the products if the quantity of circles formed is unknown. B) Using padlock probes one can only amplify and detect part of targeted sequences. C) Using padlock probes, one cannot detect or amplify the full-length genomic DNA or mRNA or cDNA. D) Additional primers are needed in order to initiate rolling circle amplification.

The recent completion of the human genome project has dramatically increased the demand for rapid, high-throughput methods for amplification, identification and quantification of specific nucleic acid sequences. Such methods should be sensitive, simple methods for amplifying and detecting nucleic acids; isothermal and homogeneous methods are preferred due to their simplicity and lower cost, extremely rapid, and adaptable to automation compared to methods requiring thermocycling. The methods should offer sensitivity while at the same time minimizing 3′ bias and the complexity of steps and reagents. Furthermore, the ability to specifically amplify RNA target from cell lysate is highly desirable. Present day technologies do not meet these demands.

Thus, there is a need for alternative amplification methods that are less prone to artifacts common to PCR based methods. Thus there is a need for simpler RCA detection and amplification methods wherein no padlock probe is required and/or additional primer sequences need not be added to the reaction. Furthermore, the RCA should be able to amplify the truly full-length targeted DNA, cDNA or mRNA with great efficiency.

The present invention addresses this need by taking advantage of the merits of rolling circle amplification, but overcoming many of the drawbacks of rolling circle amplification described previously. For instance, the methods of the present invention do not require a padlock probe to generate a circular nucleic acid for rolling circle amplification. They offer a less expensive alternative amplification method for cloning and detecting nucleic acids, and the ability to circularize and amplify full-length targeted polynucleotide sequences.

### SUMMARY OF THE INVENTION

In order to meet these needs, the present invention provides methods of detection and cloning nucleic acid molecules that take advantage of rolling circle amplification.

The invention is directed to methods of amplification, detection, and cloning of target nucleic acid molecules from complex mixtures using rolling circle amplification. The present invention includes a number of advantages that may be found in various embodiments. One advantage is to offer methods for circularizing entire target nucleic acid molecules for amplification. This allows cloning of mostly full-length target nucleic acid sequences and allows amplification and cloning of entire genomes if desired. Another advantage is to offer methods using a hairpin loop to create circular oligonucleotide molecules, instead of using padlock probes or an additional template for ligation. Another advantage in the embodiments of detection methods is the use of the target sequence itself to generate a free 3′ end to initiate rolling circle amplification. Addition primers may not be required. This allows detection without ligation in certain aspects of the invention, and few or no externally supplied primers for amplification, thus simplifying the overall reaction. Another advantage is to offer multiplex methods for detection and amplification of target polynucleotide sequences including mutation detection with circularized oligonucleotide molecules. This allows detection of mRNA or DNA without using RT-PCR or PCR, simplifying detection procedures and reducing costs.

In one aspect of the present invention, the target nucleic acid molecule is circularized without prior amplification by PCR. Free 3′ ends may be generated if needed or desired. The circularized nucleic acid molecule is then amplified by rolling circle amplification.

There are multiple embodiments of the present invention that employ different methods of circularizing the target nucleic acid without use of PCR. Generally, the target nucleic acid molecules may be circularized by a number of different methods such as ligation using enzymes (such as T4 DNA ligase) or chemical methods, photochemical reactions, site specific or homologous recombination with enzymes (such as cre-recombinase), and polymerase extensions in various forms. Circularization by recombination with an enzyme such as cre-recombinase requires attachment of specific sequences to both ends of the target nucleic acid molecule. In addition, the circularization methods of the present invention may or may not require addition of specific sequences to one or both ends of the target nucleic acid molecule in a complex mixture. The specific sequences being added to the ends of a target nucleic acid molecule are called the first linker nucleic acid molecule and the second linker nucleic acid molecule respectively. In one embodiment, a first linker nucleic acid molecule is affixed to the target nucleic acid molecule. The first linker nucleic acid comprises a sequence or moiety that allows it to be affixed to the target nucleic acid molecule. The first linker nucleic acid molecule may optionally comprise additional defined sequences that may by used later on in circularization, cloning, detection, amplification, or generation of RNA. Without limiting the generality of the foregoing, such defined sequences include restriction endonuclease sites, cre-lox cross-over sites, RNA polymerase promoter sites, polymerase termination sites, etc.

In yet another embodiment, first linker nucleic acid molecule may be affixed by hybridization to the target nucleic acid molecule or by ligation to the target nucleic acid molecule. In embodiments using hybridization, the first linker nucleic acid molecule will have a complementary region on its 3′ end for hybridization. The complementary region may be, for example, a poly-T stretch that hybridizes to the poly-A tail of mRNA. Another example is a determined sequence if the sequence of the target nucleic acid is known. If the sequence of the target nucleic acid is unknown, then the complementary region may be randomized sequences of short length such as a hexamer, a heptamer, an octamer, a nonamer, a decamer, an undecamer, or a dodecamer to allow random hybridization. In certain embodiments, the first linker nucleic acid may be extended after hybridization to the target nucleic acid molecule by addition of a polymerase such as a reverse transcriptase if the target nucleic acid is RNA. In still another embodiment, the polymerase will add specific nucleotides to the end of a nascent strand once the polymerase has reached the end of the template stand. For example, MMLV reverse transcriptase will add cytosine nucleotides to the end of the nascent strand. Such overhangs may be used directly or for extension such as oligo switch to circularize the target nucleic acid to ensure that the full-length target nucleic acid is amplified. In other embodiments, terminal transferases are used to create such overhangs. In yet other embodiments, the first linker nucleic acid molecule may comprise a pool of linkers with a random sequence at the 3′ end. Such first linker nucleic acid molecules may be used with double stranded target nucleic acid molecules. The random sequences will hybridize at the ends of the double stranded target nucleic acid molecule due to random unzipping of the ends of the double stranded target nucleic acid molecule as the nucleic acid “breathes”. Thus, such linker nucleic acid molecules may be used to circularize entire target nucleic acid molecules of unknown sequence.

In some embodiments, a second linker nucleic acid molecule is affixed to the target nucleic acid prior to or as a part of circularization of the target nucleic acid. The second linker nucleic acid molecule comprises a sequence or moiety that allows it to be affixed to the target nucleic acid molecule. The second linker nucleic acid may optionally further comprise a region complementary to the first nucleic acid molecule to enable circularization by recombination such as by the Cre-LoxP system. In another embodiment, the second linker nucleic acid molecule may have a region that can hybridize to the first linker nucleic acid molecule to allow circularization of the target nucleic acid molecule by hybridizing the first linker nucleic acid molecule to the second linker nucleic acid molecule. As with the first linker nucleic acid molecule, the second linker nucleic acid molecule may further comprise additional regions that have useful sequences such as restriction endonuclease sites, polymerase promoter sites, etc. In yet another embodiment, the second linker nucleic acid molecule is added by the oligo switch method when the target nucleic acid molecule is mRNA. This has the advantage of amplifying only full-length mRNA transcripts. For certain embodiments, a second linker nucleic acid molecule with a randomized sequence at its 3′ end can be added to the other end of the target nucleic acid by random hybridization of the second linker nucleic acid to the target nucleic acid molecule, followed by extension with polymerase.

In one aspect wherein the target nucleic acid molecule is mRNA the circularized nucleic acid molecule ideally includes full-length cDNAs. The circularized full-length cDNA may be amplified with randomers as primers to generate multiple copies of full-length double strand cDNA. In some embodiments, the circularized nucleic acid molecule will include an RNA polymerase promoter sequence such as the T7 RNA polymerase promoter. Depending upon the orientation and position of the T7 promoter, the amplified DNA can be used as a template to generate multiple copies of antisense RNA (aRNA) or of mRNA. By combining RNA transcription with rolling circle amplification, the aRNA or mRNA amplification efficiency is greatly enhanced compared to use of T7 polymerase in the absence of amplification. Furthermore, the methods of the present invention may be designed to eliminate the 3′ bias and simplify the whole amplification process. In still other embodiments, RNA polymerase promoters may be provided at both ends of the target nucleic acid molecule, incorporated into the circularized nucleic acid molecule in order to generate double stranded RNA.

The target nucleic acid molecule may be circularized by a number of methods including, without limitation, blunt end ligation, annealing complementary ends followed by ligation, recombination between complementary regions, or annealing a primer with polymerase extension. The circularization will result in at least one strand of the nucleic acid being circularized. Circularization of an mRNA target nucleic acid molecule may be performed by self-priming after the reverse transcriptase to synthesize the second strand of the cDNA followed by closing the circle by self-ligation. A hairpin loop structure at the 5′ end of the first strand cDNA will further assist the self-ligation reaction. The product of such self-primed synthesis of the second strand is a double stranded cDNA molecule closed at the terminus corresponding to the 5′ terminus of the mRNA by a hairpin loop. In the same manner, self-priming can also be used to circularize single strand DNA.

The present invention encompasses multiple methods of rolling circle amplification after the target nucleic acid molecule has been circularized. In some embodiments, a polymerase that can initiate at an appropriate promoter sequence is used. The promoter sequence may have been added in the first linker nucleic acid, the second linker nucleic acid, or the combination of the two. In certain embodiments, the polymerase needs a free 3′ end to begin polymerization. Such free 3′ end may be generated by a number of methods. In one embodiment, the 3′ end results from the circularization. In another embodiment, the 3′ end is generated after circularization by addition of one or more primers that hybridize to some portion of the circularized nucleic acid. In certain embodiments, the primers may be RNA:DNA chimeras. In some embodiments, randomers can be used as primers. In still other embodiments, the free 3′ end is introduced by nicking the circularized DNA randomly with limited amounts of endonucleases. In the case of amplification of an RNA, the RNA may be nicked with limiting amounts of RNaseH or the RNA may be completely removed with excess RNaseH. In still other embodiments, the free 3′ end is introduced by cutting with a restriction endonuclease at a hemi-methylated restriction site.

With a free 3′ end, the target nucleic acid may be amplified by rolling circle amplification in such embodiments needing a free 3′ end. Some embodiments include generation of an RNA transcript by adding an RNA polymerase that initiates transcription from a promoter added to the target nucleic acid molecule. In some embodiments, a single initiation point is used which results in linear amplification. This may be achieved through addition of a single primer, use of a single polymerase start point, or other generation of free 3′ ends on only one strand of the circularized nucleic acid molecule. In other embodiments, exponential amplification will be achieved by generation of free 3′ ends corresponding to both strands. An example would be to add a pair of primers each of which anneals to different strands of the circularized nucleic acid molecule.

In yet another embodiment, the circularized full-length targeted polynucleotide sequences can be constructed to contain necessary components so that they can be used to express proteins in vivo or vitro. Such methods eliminate the complexity of inserting double strand cDNA into a vector or plasmid.

In yet another aspect of the present invention, a target nucleic acid molecule is detected by addition of a circular nucleic acid molecule that comprises a first region that will hybridize to the target nucleic acid molecule. The target nucleic acid molecule is hybridized to the circular nucleic acid molecule, and rolling circle amplification is initiated at an extendable free 3′ end of the target nucleic acid molecule. The extendable free 3′ end may be generated in the target nucleic acid molecule before or after the target nucleic acid molecule has been hybridized to the circular nucleic acid molecule. This is particularly important when the target nucleic acid molecule is mRNA which has a poly-A tail at the 3′ end. The extendable free 3′ end may be generated by cleaving the target nucleic acid molecule prior to hybridization or after hybridization by site specific cleavage or by random nicking of the target nucleic acid molecule. One of skill in the art is aware of many methods of site specific cleavage, which are included in the present invention. Examples include restriction endonucleases and, in the case of RNA, ribozymes, RNAi Dicer, etc. Random nicking may be performed with chemical agents or non-specific nucleases.

In one embodiment of the present invention, the circular nucleic acid molecules can be constructed by using synthetic oligonucleotide with self-ligation, instead of template dependent ligation or by using a padlock probe. Such a method is a single molecular reaction or intramolecular reaction, which is more efficient and accurate compared to the use of padlock probes or template dependent ligation reactions.

In another embodiment of the present invention, the full-length circular nucleic acid molecules of any gene can be constructed by using an existing full-length cDNA clone library with PCR amplification. The resulting full-length circular nucleic acid molecules can be used to amplify, detect and quantify specific genes.

In yet another embodiment, free 3′ ends can be selectively generated by RNaseH or other methods such that free 3′ ends are only generated in circularized nucleic acid molecules which have a particular sequence, such as a mutation or lack thereof. Consequently rolling circle amplification will only be initiated on circularized nucleic acid molecules with the specific sequence. Compared to RT-PCR or TaqMan, such methods offer much better accuracy and simplicity.

Once rolling circle amplification has been initiated at a free 3′ end, additional primers complimentary to the nascent strand may be used to further enhance amplification.

In yet another aspect of the present invention, mutations such as single nucleotide polymorphisms in the target nucleic acid molecules can be detected by selectively generating free 3′ ends only in the mutant or non-mutant target nucleic acid molecule. Examples include ribozymes targeted at the site of the mutation, and hybridization of the target nucleic acid molecule with nucleic acid molecules complementary to the target nucleic acid molecule with or without the mutation followed by nicking with enzymes such as S1 nuclease that will cleave at mismatches.

In yet another aspect of the present invention, the target molecules can be single strand or double strand DNA. A fragment of RNA can be added where the 3′ extension has been blocked. If the targeted nucleic acid molecules is present, the RNA fragment will hybridize to the target and then any enzymes such as Rnase H will digest the added fragment of RNA to generate the free 3′ end. Thereafter the added circular nucleic acid molecules will initiate the rolling circle amplification. The RNA fragment may contain a hairpin loop to increase the reaction specificity.

In yet another aspect of the present invention, the target molecules can be single strand or double strand DNA. A fragment of DNA-RNA chimeric fragment can be added where the 3′ extension has been blocked. If the targeted nucleic acid molecules is present, the DNA-RNA fragment will hybridize to the target, and then enzymes such as Rnase H will digest the added fragment of DNA-RNA chimeric to generate the free 3′ end. Thereafter the added circular nucleic acid molecules will initiate rolling circle amplification. The DNA-RNA chimeric fragment may contain a hairpin loop to increase the reaction specificity.

Detection of the amplified product may be performed by any method applicable to the detection of nucleic acids, such as those described below.

### BRIEF DESCRIPTION OF THE FIGURES

FIG. 1. depicts synthesis of circular nucleic acid molecules with self-ligation probes.

FIG. 2. depicts synthesis of circular nucleic acid molecules from full-length cDNA clone library.

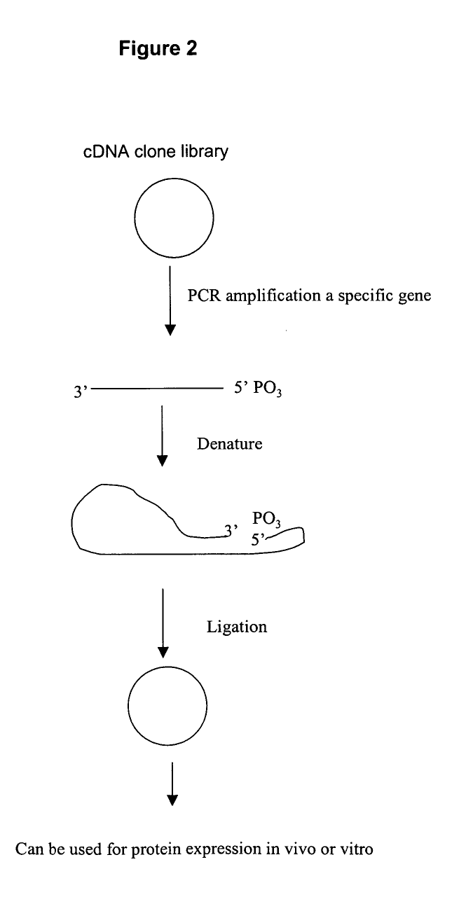

FIG. 3. depicts detection of a target nucleic acid molecule with a circular nucleic acid molecule.

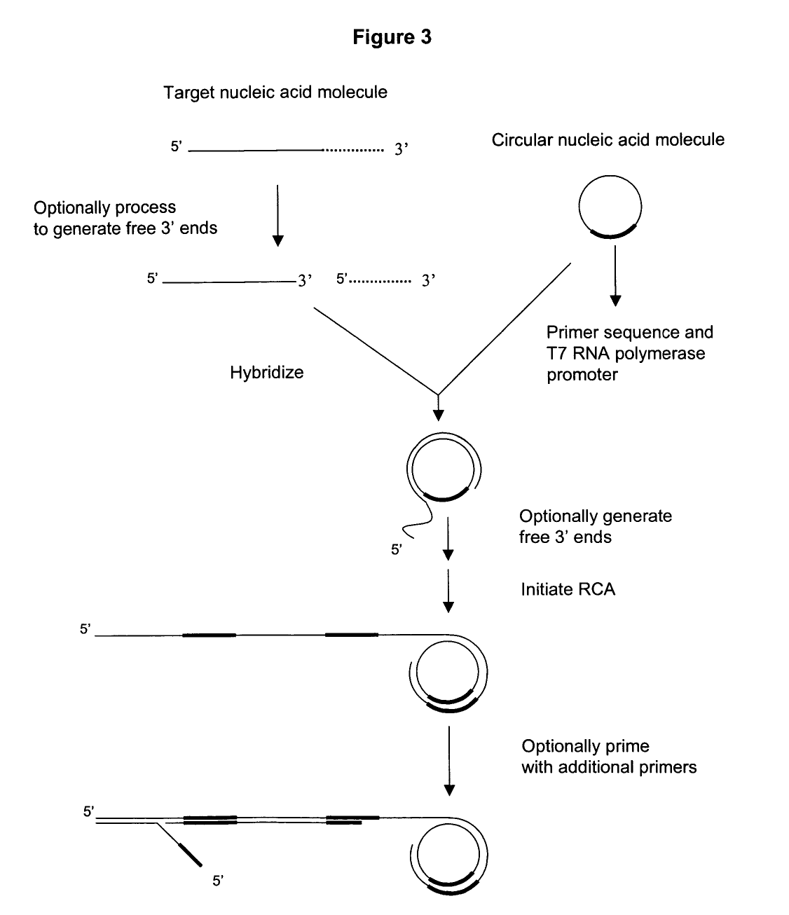

FIG. 4. depicts detection and amplification of a target RNA molecule with a circular nucleic acid molecule.

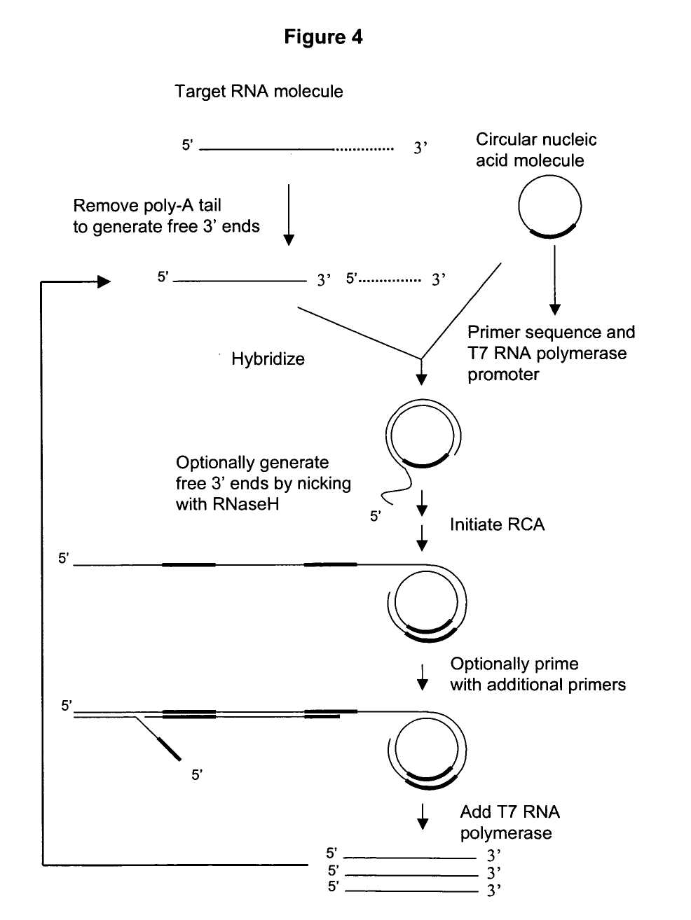

FIG. 5. depicts detection and amplifcation of a target RNA molecule with circular nucleic acid molecules synthesized from full-length clone library.

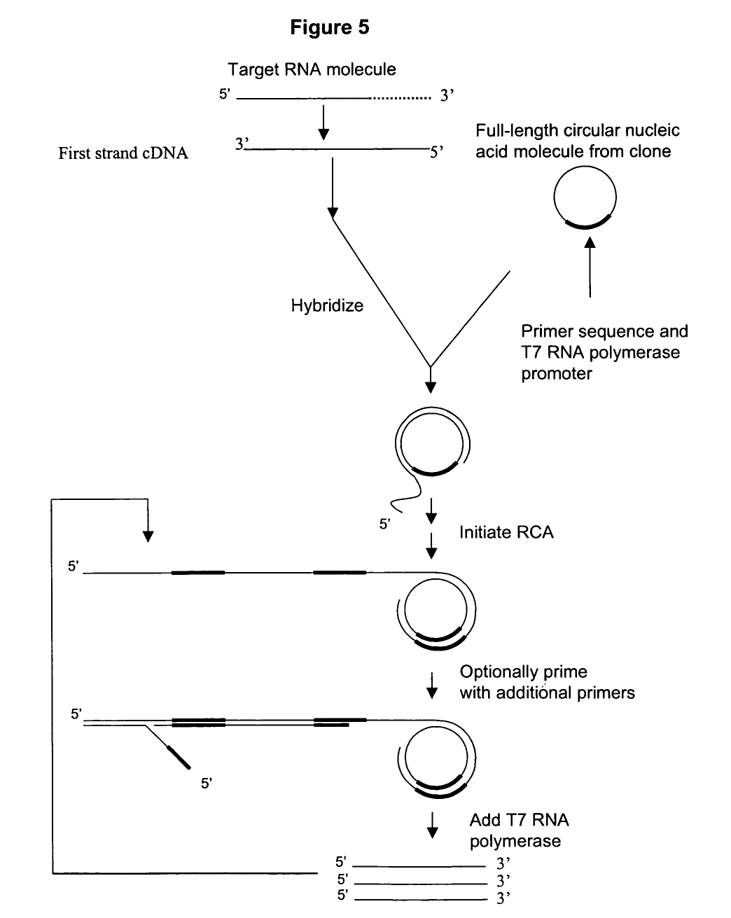

FIG. 6. depicts detection and amplification of a target nucleic acid molecule with circular nucleic acid molecules containing randomer sequences.

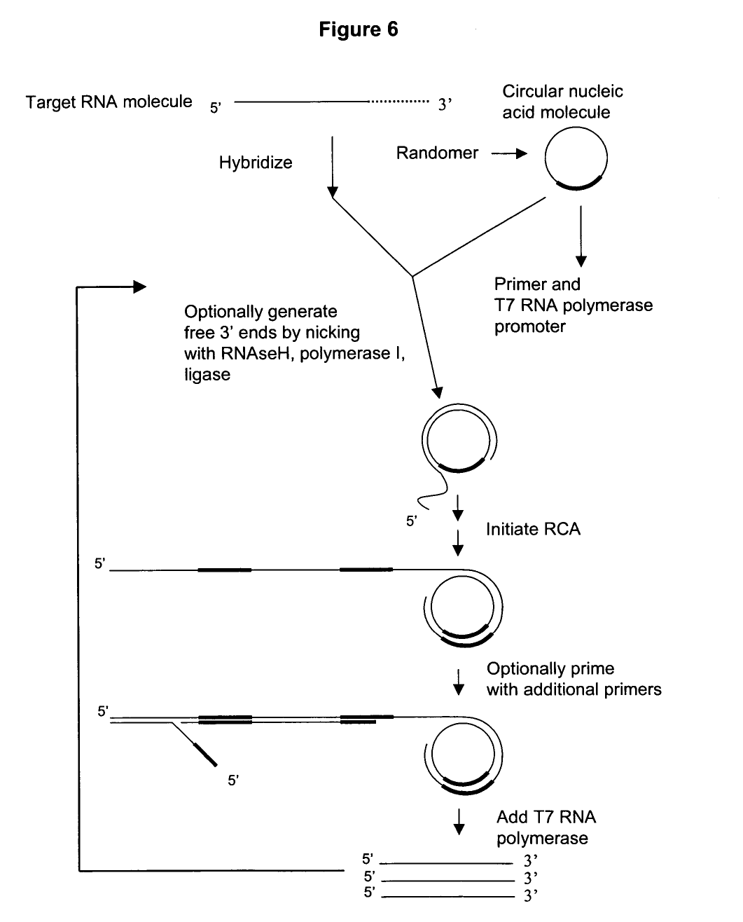

FIG. 7. depicts detection and amplification of a target RNA molecule with a circular nucleic acid molecule wherein the free 3′ ends are generated by either complete digestion with RNaseH or nicking with RNaseH.

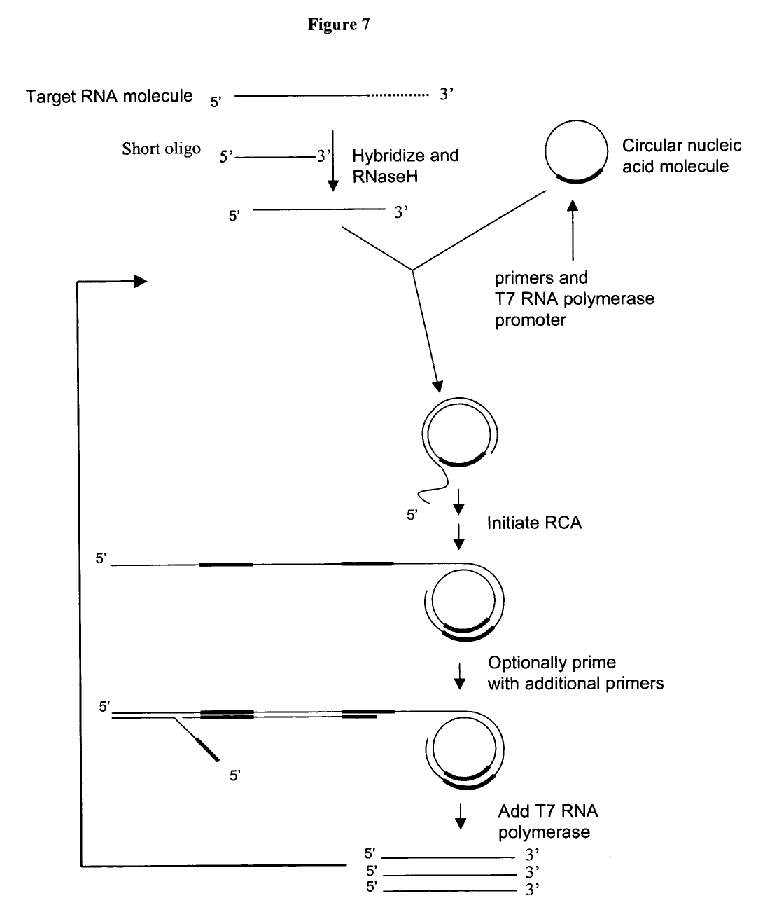

FIG. 8. depicts detection and amplification of a target single or double strand DNA with a circular nucleic acid molecule wherein the free 3′ ends are generated by adding short RNA fragment digested or nicked with Rnase H.

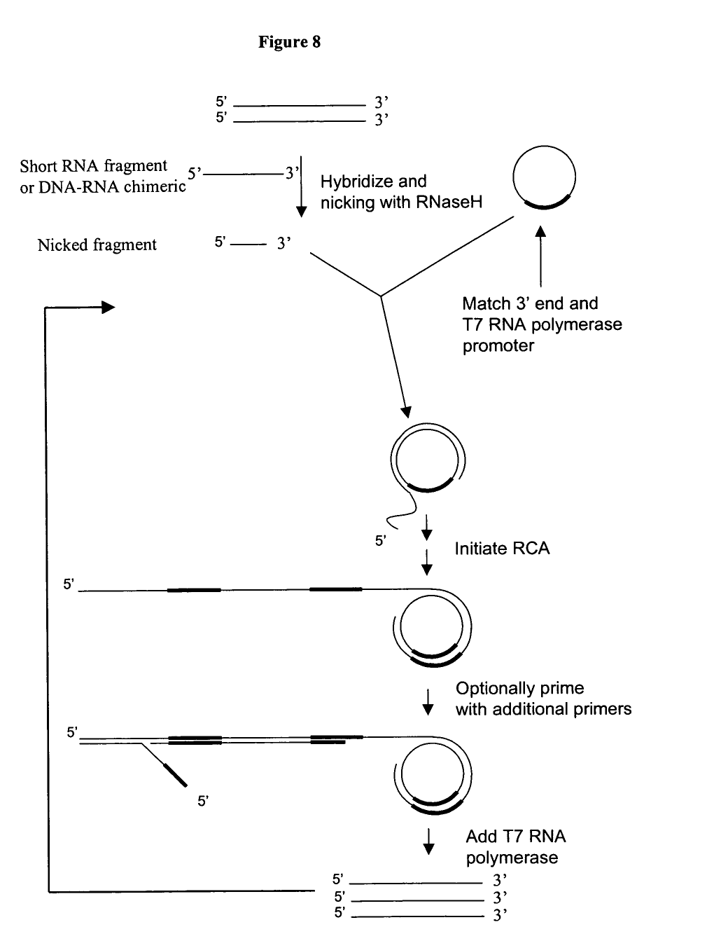

FIG. 9. depicts detection and amplification of a target nucleic acid molecule with an open circular nucleic acid molecule wherein the free 3′ ends are provided initially from the target nucleic acid molecule.

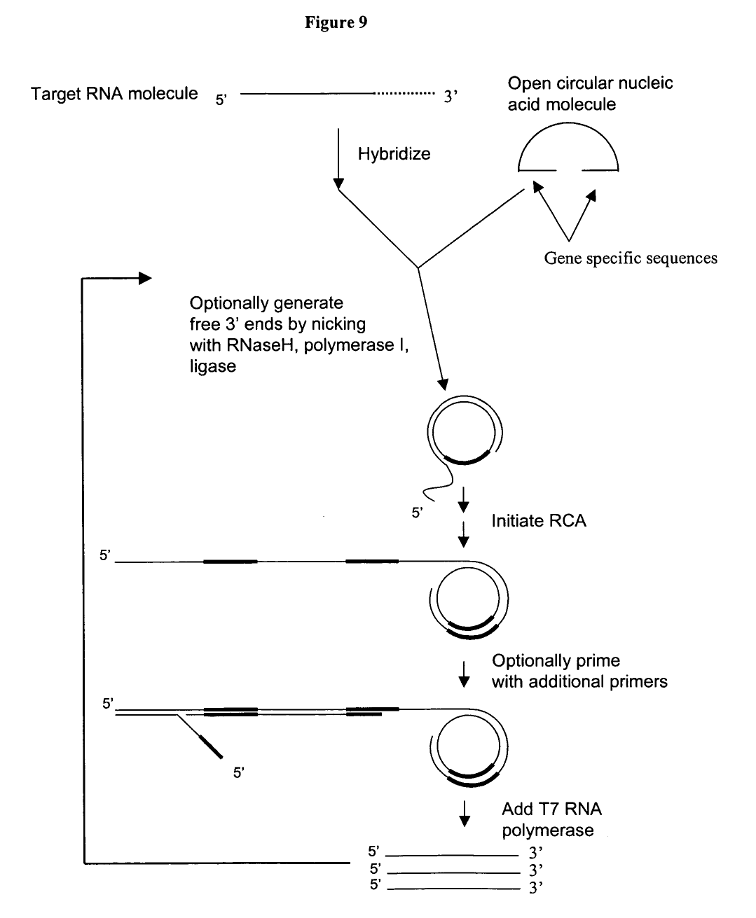

FIG. 10. depicts a general method of amplification of a target nucleic acid by circularization.

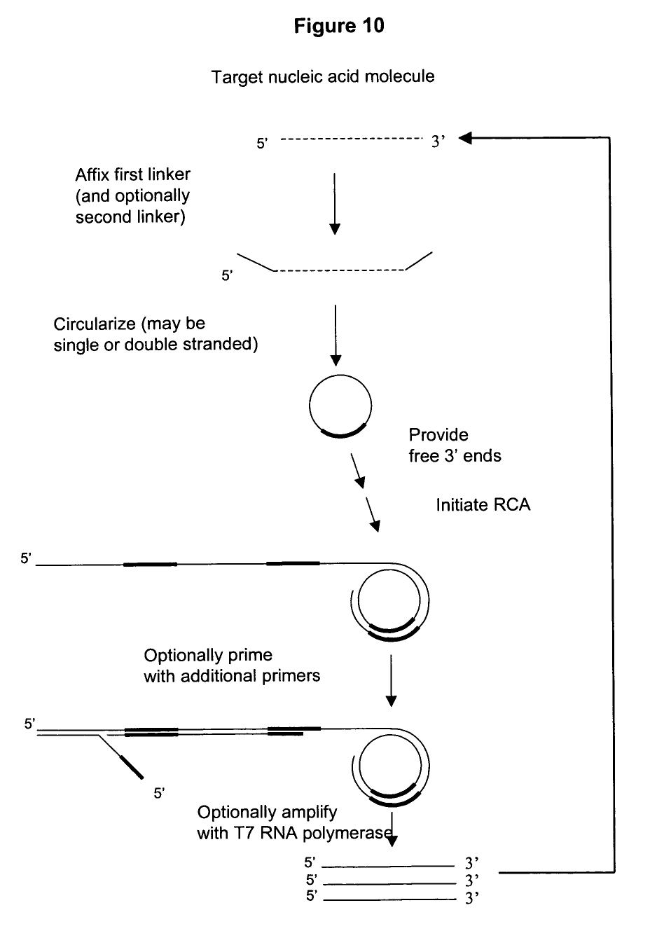

FIG. 11. depicts amplification of a target nucleic acid by circularization with a first and second linker nucleic acid molecule, where one linker is an switch oligonucleotide.

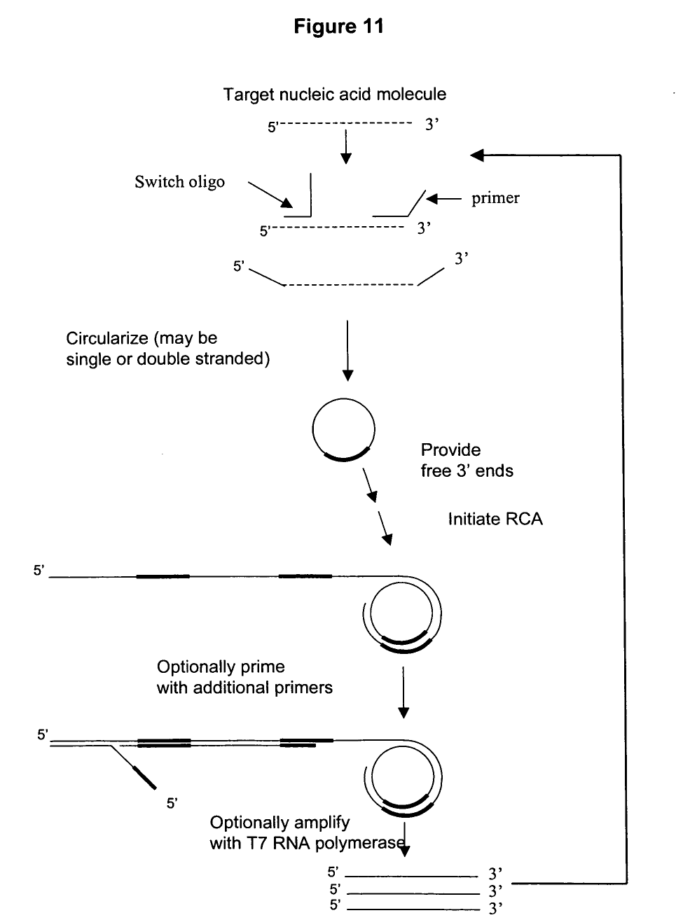

FIG. 12. depicts amplification of a target nucleic acid by circularization with a first linker.

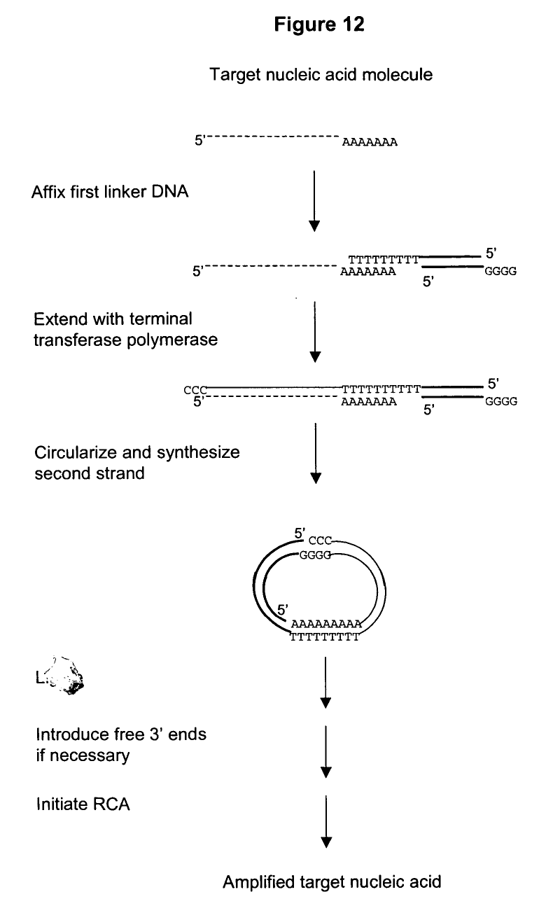

FIG. 13. depicts amplification of a target nucleic acid by circularization with a first linker nucleic acid molecule optionally containing a hairpin loop and self-priming at the end of the target nucleic acid molecule.

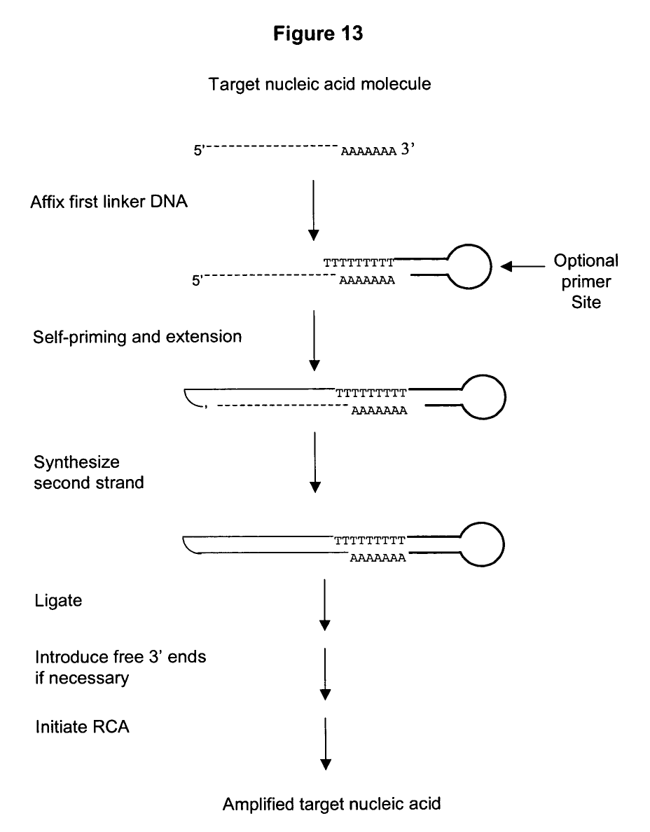

FIG. 14. depicts amplification of a target nucleic acid by circularization with a first linker nucleic acid molecule and a second linker nucleic acid molecule with a randomer sequence used as an oligo switch template.

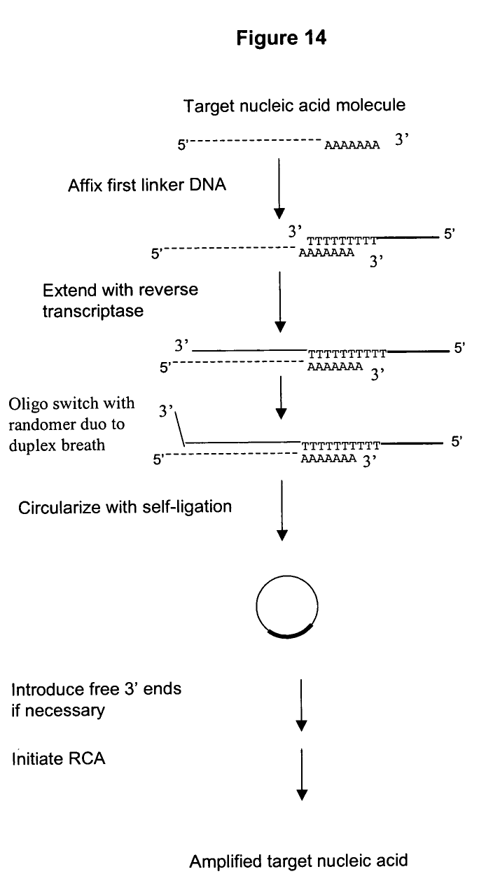

FIG. 15. depicts amplification of a target nucleic acid by circularization with a first linker and second linker by recombination.

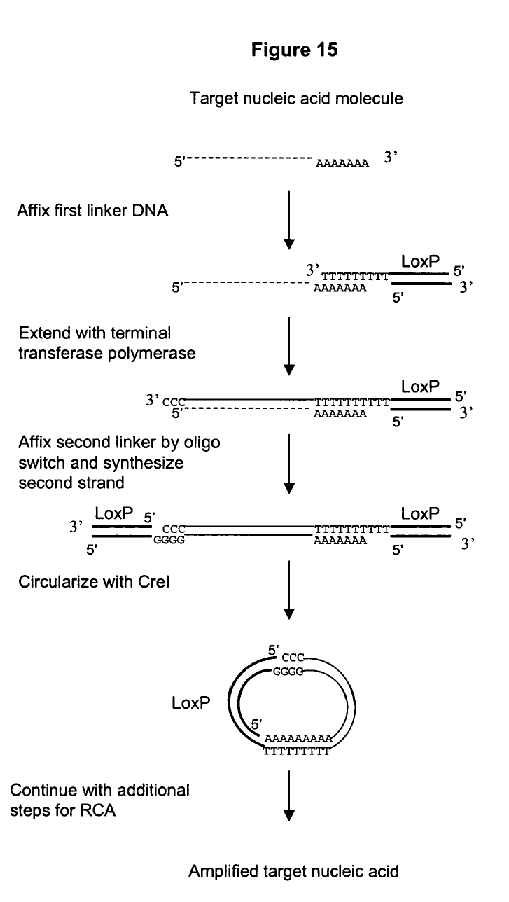

FIG. 16. depicts amplification of a target double strand nucleic acid by rolling circle amplification.

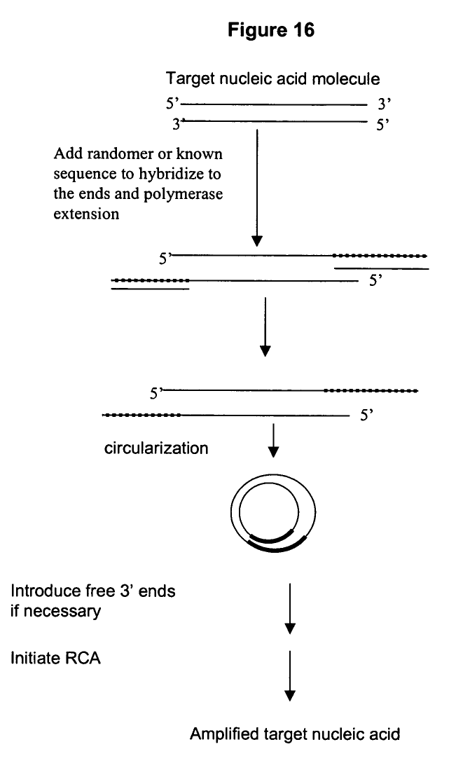

### DETAILED DESCRIPTION OF EMBODIMENTS OF THE INVENTION

Definitions

As used herein, a “circular nucleic acid molecule” is a nucleic acid molecule with at least one contiguous strand. The circular nucleic acid molecule is used to detect and amplify a target nucleic acid molecule. At least a portion of the target nucleic acid molecule will be contained within or complementary to a portion of the circular nucleic acid molecule. The circular nucleic acid molecule may be RNA, DNA, PNA, or any combination thereof. The circular nucleic acid molecule may contain any natural or unnatural bases and may have missing bases. The circular nucleic acid molecule may be generated by any suitable techniques, including without limitation, synthetic and natural methods.

As used herein, a “circularized nucleic acid molecule” is a nucleic acid molecule that contains or is complementary to the target nucleic acid sequence within the circular portion. The circularized nucleic acid molecule is generated as a part of the amplification and cloning process. The circularized nucleic acid molecule comprises at least one contiguous strand. The circularized nucleic acid molecule may be RNA, DNA, PNA, or any combination thereof. The circularized nucleic acid molecule may contain any natural or unnatural bases and may have missing bases.

As used herein, a “free 3′ end” is a 3′ end of a nucleic acid molecule that is annealed to a template nucleic acid strand that a polymerase may extend.

As used herein, a “target nucleic acid molecule” is the nucleic acid molecule to be cloned or detected through rolling circle amplification. The target nucleic acid molecule may be obtained from any source. It can be mRNA, rRNA, RNAi, RNA being processed, genomic DNA, cDNA, etc.

Preparation of the Target Nucleic Acid

The present invention includes target nucleic acid molecules that are to be amplified for cloning, detection, etc. The disclosed methods may be adapted to any nucleic acid molecule of interest. The nucleic acid may be obtained from any source including, without limitation, cellular or tissue samples, nucleic acid molecules in libraries, chemically synthesized nucleic acid molecules, genomic nucleic acid molecules, cloned nucleic acids, mixtures of such nucleic acids, messenger RNAs, including splice variants and intermediates. The methods are particularly suited to generating libraries from mixtures of mRNAs. The only requirement is that the nucleic acid be amenable to circularization according to the methods of the present invention or have a defined sequence for detection by the methods of the present invention.

The nucleic acids may be obtained by a wide range of methods available to one of skill in the art. Detailed protocols for numerous such procedures are described in, e.g., in Ausubel et al. Current Protocols in Molecular Biology (Supplemented through 2000) John Wiley & Sons, New York; Sambrook et al. Molecular Cloning—A Laboratory Manual (2nd Ed.), Vol. 1-3, Cold Spring Harbor Laboratory, Cold Spring Harbor, N.Y., 1989, and Berger and Kimmel Guide to Molecular Cloning Techniques, Methods in Enymology volume 152 Academic Press, Inc., San Diego, Calif.

Once the nucleic acid molecules of interest have been obtained, the molecules may be further manipulated depending upon the later methods applied to the molecules. For example, when detecting a target nucleic acid molecule with a circular nucleic acid, the target nucleic acid may be pre-treated to generate different or additional free 3′ ends. Examples include targeted cleavage with a site-specific ribozyme or hybridization to a complementary nucleic acid sequence and digestion with the appropriate nuclease. In the case of mRNA, the poly-A tail may be removed by any suitable technique known to one of ordinary skill in the art. An example for mRNA would be to add single stranded polyT DNA and RNAseH. In addition, longer nucleic acid molecules may be cleaved to generate shorter fragments. Examples of such cleavage include physical shearing of the DNA by pipetting or sonication, digestion with restriction endonucleases, etc.

In addition, to assist in circularization, the nucleic acid may be treated with a variety of other protocols such as filling in over-hangs generated by restriction enzymes, adding phosphate groups with poly-nucleotide kinases for later ligation or removing phosphate groups with phosphatases to prevent later ligation. The cohesive ends generated by restriction endonucleases may be annealed and ligated to circularaize the nucleic acid for rolling circle amplification. As discussed above, one of skill in the art may find detailed protocols for all such procedures in the literature.

Circularization of the Target Nucleic Acid

For amplification without PCR, the target nucleic acid must be circularized. The present invention includes circularization by ligation, hybridization and ligation, recombination, and chemical reaction. For ligation, any ligase will be suitable. One of skill in the art will be able to select an appropriate ligase for the particular reaction: DNA ligases for DNA nucleic acids, RNA ligases for RNA, etc.

One of skill in the art is aware of many suitable ligases, such as T4 RNA ligase to circularize single strand DNA or RNA and T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J Biol. Chem. 253:4590-4592 (1978)), AMPLIGASE.RTM. (Kalin et al., Mutat Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is preferred for ligations involving RNA target nucleic acid molecules due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

Circularization of the target nucleic acid molecule may include addition of a first linker nucleic acid molecule. The first linker nucleic acid molecule may be affixed to the target nucleic acid molecule by a range of techniques known to those of ordinary skill in the art. It is preferred that the first linker nucleic acid molecule be affixed to an end of the target nucleic acid. In certain embodiments, a first linker nucleic acid molecule may be affixed at both ends of the target nucleic acid. Methods of affixing the first nucleic acid to the molecule may include ligation, hybridization and ligation, and hybridization followed polymerase extension and other enzymatic reactions such as terminal transferase. A preferred example is a first linker comprising a poly-T sequence at its 3′ end. In addition, the linker may comprise one or more of a number of other sequences that may be useful for circularization, detection, etc. Examples include RNA and/or DNA polymerase promoters, site specific recombination sequences such as loxP, homologous sequences for general recombination, restriction sites (including hemimethylated sites), transcription termination sites, ribosome binding sites, ribozymes, RNAi, replication origins, genes, etc.

Optionally, a second linker may be affixed by methods similar to those above. The second linker may comprise one or more of a number of other sequences that may be useful for circularization, detection, etc. In addition, when the target nucleic acid molecule is mRNA, the second linker may be affixed by the CAPswitch method, which is described in U.S. Pat. No. 5,962,271, which is herein incorporated by reference. In addition, the second linker may be affixed by the oligo-capping method described in U.S. Pat. No. 5,597,713, which is herein incorporated by reference. The second linker may also be affixed by hybridization in a manner that facilitates template switching, as described by Patel et al. PNAS, 93:2969-2974.

For double stranded nucleic acid molecules, a first and optionally a second linker nucleic acid molecule may comprise random sequences if the target sequence is unknown, or defined sequences if the target sequence is known at their 3′ ends which can hybridize to the target nucleic acid molecule and be extended by addition of an appropriate polymerase. The linker nucleic acid molecules may comprise additional sequences for circularization. The first linker or optionally the second linker will hybridize at the ends of double stranded nucleic acid molecule due to random unzipping of the double helix at the ends from breathing of the duplex. Once the strand has opened and the linker has hybridized, a polymerase may extend from the free 3′ end of the target nucleic acid molecules to generate blunt ends. The resulting double strand targeted nucleic acid molecules can be then circularized and amplified.

The target nucleic acid molecule may be circularized by hybridization. The affixed first linker may hybridize to the other end of the target nucleic acid molecule or to an optionally affixed second linker. One of skill in the art will recognize that the present invention may optionally include additional intervening linkers as desired. Once hybridized, ligase should be used to generate at least one strand that has been ligated into a contiguous molecule.

The target nucleic acid molecule may also be circularized by chemical reaction or photo-reaction.

Additionally, mRNA target nucleic acid molecules may be circularized by self-priming of the reverse transcription reaction to generate the second DNA strand. Once the strand has been synthesized, the circle may be closed by ligation. In some embodiments, the first linker nucleic acid will have a hairpin to enhance circularization after self-priming. The hairpin loop may be of arbitrary size and can accommodate any additional sequence elements that may be desirable.

Additionally, the first strand cDNA synthesis from mRNA may be not full-length. In such case, a first and optionally a second linker nucleic acid molecule may comprise random sequences at their 3′ ends which can hybridize to the 3′ end first strand cDNA and be extended by addition of an appropriate polymerase. The first strand cDNA can be synthesized with modified polyT or modified random primers. The linker nucleic acid molecules may comprise additional sequences for circularization. The random sequences will hybridize at the 3′ end of first strand cDNA due to random unzipping of the RNA: DNA duplex at the 3′ end of the first strand from breathing of the duplex. Once the strand has opened and the linker has hybridized, the first strand cDNA may be further extended from the 3′ end. Optionally, mRNA can be digested. The randomer will hybridize to the 3′ end of the first strand cDNA and then the first strand cDNA can be further extended.

In addition, the target nucleic acid may be circularized by recombination. Such recombination may be accomplished with a site-specific recombinase such as Cre or through a recombinase that will recombine molecules with homologous sections. Recombination with LoxP-CreI is described in U.S. Pat. No. 5,591,609, which is hereby incorporated by reference.

Generation of Free 3′ Ends for Polymerase Extension

In order to begin rolling circle amplification, most polymerases need a free 3′ end. For amplification with such polymerases, a free 3′ end may need to be generated or supplied. For methods that involve circularization of the target nucleic acid, the circularization itself may result in free 3′ ends. Many methods are available for generating free 3′ ends if needed. Where the target nucleic acid is an RNA molecule, and it is hybridized to a complementary DNA molecule, RNAseH may be used to digest the RNA molecule entirely, or limiting amounts of RNAseH may be used to nick the RNA and generate free 3′ ends. Furthermore, limiting amounts of any endonuclease may be used to nick double stranded nucleic acid molecules. One of skill in the art will appreciate that the limiting amount will need to be controlled such that both strands are not nicked because at least one strand must remain an intact circle for rolling circle amplification. Similarly, limiting amounts of chemicals that nick DNA may be used to generate free 3′ ends.

In addition, specific free 3′ ends may be generated by use of thiophosphorylated or hemimethylated double stranded nucleic acids. Certain restriction endonucleases will cut only one strand when the restriction site is thiophosphorylated or hemimethylated. Such hemimethylated DNA may be generated by a number of methods. For example, nucleic acid molecules may be chemically synthesized with methylated nucleotides at key positions; the nucleic acid molecule may be methylated with site specific DNA methylases in vitro; or the nucleic acid molecule may be obtained from an organism that expresses the requisite site-specific DNA methylase. Also, certain restriction endonucleases may be used that naturally only cut one strand of a duplex, e.g., N.Alw I, N.BstNB I (both available from New England Biolabs).

Additionally, ribozymes or RNAi constructs such as Dicer may be used to cleave the ribonucleic acid molecules at specific locations, thus generating free 3′ ends.

Another method of generating free 3′ ends is by supplying an oligonucleotide primer. A preferred primer is a strand displacement primer. One form of strand displacement primer is an oligonucleotide having sequence complementary to a strand of a circular nucleic acid. This sequence is referred to as the matching portion of the strand displacement primer. The matching portion of a strand displacement primer may be complementary to any sequence. However, it is preferred that it not be complementary to any additional strand displacement primers, if such are being used. This prevents hybridization of the primers to each other. The matching portion of a strand displacement primer may be complementary to all or a portion of the inserted nucleic acid molecule, although this is not preferred. The matching portion of a strand displacement primer can be any length that supports specific and stable hybridization between the primer and its complement. Generally this is 12 to 35 nucleotides long, preferably 18 to 25 nucleotides long.

It is preferred that strand displacement primers also contain additional sequence at their 5′ end that does not match any part of a strand of the circular nucleic acid. This sequence is referred to as the non-matching portion of the strand displacement primer. The non-matching portion of the strand displacement primer, if present, serves to facilitate strand displacement during DNA replication. The non-matching portion of a strand displacement primer may be any length, but is generally 1 to 100 nucleotides long, and preferably 4 to 8 nucleotides long.

Optionally, the strand displacement primers may also contain additional RNA sequence at the 5′ end of that may or may not match any part of a strand of the circular nucleic acid. Examples of use of such chimeric primers are disclosed in U.S. Pat. No. 6,251,639 and U.S. Patent Application 2003/0087251, both of which are hereby incorporated by reference.

Additional strand displacement primers may be used to increase the amplification of the target nucleic acid. The additional strand displacement primers may be complementary to the same strand that the first strand displacement primer complements to linearly increase the amplification, or have the same sequence as the strand that the first strand displacement primer complements to geometrically increase the amplification. Again, it is preferred that no primer strand displacement primer is complementary to any other strand displacement primer to prevent the primers from hybridizing to one another.

Strand displacement primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated linear vectors that might otherwise interfere with hybridization of probes and primers to the amplified nucleic acid. Strand displacement primers can be used for strand displacement replication and strand displacement cascade amplification, both described below.

Additionally, the free 3′ ends may be provided by addition of short oligonucleotides of random sequence. The preferred length is hexamers. To assist strand displacement, the nucleotides at the 5′ end may be RNA.

Amplification by Rolling Circle Amplification

Rolling circle amplification may be performed with the circularized nucleic acid molecules and circular nucleic acid molecules of the present invention. This reaction requires the two components: (a) a free 3′ end, and (b) a rolling circle polymerase. The polymerase catalyzes primer extension and strand displacement in a processive rolling circle polymerization reaction that proceeds as long as desired, generating a molecule of up to 100,000 nucleotides or larger. This reiterated DNA sequence DNA (R-DNA) consists of long repeats of the circular or circularized nucleic acid molecule sequence. A number of references disclose primer, primer design, and amplification techniques, including U.S. Pat. Nos. 5,871,921, 5,648,245, 5,866,377 and 5,854,033, all hereby incorporated by reference.

During rolling circle replication one may additionally include radioactive, or modified nucleotides such as bromodeoxyuridine triphosphate, in order to label the DNA generated in the reaction. Alternatively, one may include suitable precursors that provide a binding moiety such as biotinylated nucleotides (Langer et al., Proc. Natl. Acad. Sci. USA 78:6633 (1981)).

Strand displacement replication is a way to amplify R-DNA. Strand displacement replication is accomplished by hybridizing strand displacement primers to R-DNA and allowing a DNA polymerase to synthesize DNA from these primed sites. The product of strand displacement replication is referred to as secondary reiterated sequence DNA or R-DNA-2. Strand displacement replication can be accomplished by performing rolling circle replication to produce R-DNA, and then mixing strand displacement primer with the R-DNA and incubating to replicate the reiterated sequence DNA. The strand displacement primer is complementary to a part of the circular or circularized nucleic acid molecule used to generated R-DNA as described earlier.

Strand displacement replication can also be carried out simultaneously with rolling circle replication. This is accomplished by mixing strand displacement primer with the circular or circularized nucleic acid molecule and rolling circle replication primer prior to incubating the mixture for rolling circle replication. For simultaneous rolling circle replication and strand displacement replication, it is preferred that the rolling circle DNA polymerase be used for both replications. This allows optimum conditions to be used and results in displacement of other strands being synthesized downstream. Generally, strand displacement replication can be performed by, simultaneous with or following rolling circle replication, mixing a strand displacement primer with the R-DNA and incubating to replicate the reiterated sequence DNA to result in the formation of secondary reiterated sequence DNA.

To optimize the efficiency of strand displacement replication, it is preferred that a sufficient concentration of strand displacement primer be used to obtain sufficiently rapid priming of the growing R-DNA strand to out-compete any remaining unligated linear nucleic acid molecules that might be present for binding to R-DNA. In general, this is accomplished when the strand displacement primer is in very large excess compared to the concentration of single-stranded sites for hybridization of the strand displacement primer on R-DNA. Optimization of the concentration of strand displacement primer can be aided by analysis of hybridization kinetics using methods such as those described by Young and Anderson, “Quantitative analysis of solution hybridization” in Nucleic Acid Hybridization: A Practical Approach (IRL Press, 1985) pages 47-71. Alternatively, the efficiency of strand displacement replication can be improved by the removal of unligated linear nucleic acid molecules prior to amplification of the R-DNA. In strand displacement replication, it is preferred that the concentration of strand displacement primer generally be from 500 nM to 5000 nM, and most preferably from 700 nM to 1000 nM.

As a strand displacement primer is elongated, the DNA polymerase will run into the 5′ end of the next hybridized strand displacement molecule and will displace its 5′ end. In this fashion a tandem queue of elongating DNA polymerases is formed on the R-DNA template. As long as the rolling circle reaction continues, new strand displacement primers and new DNA polymerases are added to R-DNA at the growing end of the rolling circle.

When strand displacement replication is carried out in the presence of a tertiary strand displacement primer, an exponential amplification of R-DNA sequences takes place. This special and preferred mode of strand displacement replication is referred to as strand displacement cascade amplification (SDCA). In SDCA, a strand displacement primer primes replication of R-DNA to form R-DNA-2, as described above. The tertiary strand displacement primer can then hybridize to, and prime replication of, R-DNA-2 to form R-DNA-3 (tertiary reiterated sequence DNA). Strand displacement of R-DNA-3 by the adjacent, growing R-DNA-3 strands makes R-DNA-3 available for hybridization with secondary strand displacement primer. This results in another round of replication resulting in R-DNA-4 (which is equivalent to R-DNA-2). R-DNA-4, in turn, becomes a template for DNA replication primed by tertiary strand displacement primer. The cascade continues in this manner until the reaction stops or reagents become limiting. This reaction amplifies DNA at an almost exponential rate, although kinetics are not truly exponential because there are stochastically distributed priming failures, as well as steric hindrance events related to the large size of the DNA network produced during the reaction.

In a preferred mode of SDCA, the rolling circle replication primer serves as the tertiary strand displacement primer, thus eliminating the need for a separate primer. For this mode, the rolling circle replication primer should be used at a concentration sufficiently high to obtain rapid priming on the growing R-DNA-2 strands. To optimize the efficiency of SDCA, it is preferred that a sufficient concentration of secondary strand displacement primer and tertiary strand displacement primer be used to obtain sufficiently rapid priming of the growing R-DNA strand to out-compete R-DNA for binding to its complementary R-DNA, and, in the case of secondary strand displacement primer, to out-compete any remaining unligated linear nucleic acid molecule that might be present for binding to R-DNA. In general, this is accomplished when the secondary strand displacement primer and tertiary strand displacement primer are both in very large excess compared to the concentration of single-stranded sites for hybridization of the strand displacement primers on R-DNA. For example, it is preferred that the secondary strand displacement primer is in excess compared to the concentration of single-stranded secondary strand displacement primer complement sites on R-DNA, R-DNA-3, R-DNA-5, and so on. In the case of tertiary strand displacement primer, it is preferred that the tertiary strand displacement primer is in excess compared to the concentration of single-stranded tertiary strand displacement primer complement sites on R-DNA-2, R-DNA-4, R-DNA-6, and so on. Such an excess generally results in a primer hybridizing to its complement in R-DNA before amplified complementary R-DNA can hybridize. Optimization of primer concentrations can be aided by analysis of hybridization kinetics (Young and Anderson). In a strand displacement cascade amplification, it is preferred that the concentration of both secondary and tertiary strand displacement primers generally be from 500 nM to 5000 nM, and most preferably from 700 nM to 1000 nM.

As in the case of secondary strand displacement primers, if the concentration of DNA polymerase is sufficiently high, the polymerase will initiate DNA synthesis at each available 3′ terminus on the hybridized tertiary strand displacement primers, and these elongating R-DNA-3 molecules will block any hybridization by R-DNA-2. As a tertiary strand displacement primer is elongated to form R-DNA-3, the DNA polymerase will run into the 5′ end of the next hybridized tertiary strand displacement primer molecule and will displace its 5′ end. In this fashion a tandem queue of elongating DNA polymerases is formed on the R-DNA-2 template. As long as the reaction continues, new rolling circle replication primers and new DNA polymerases are added to R-DNA-2 at the growing ends of R-DNA-2. This hybridization/replication/strand displacement cycle is repeated with hybridization of secondary strand displacement primers on the growing R-DNA-3.

Generally, strand displacement cascade amplification can be performed by, simultaneous with, or following, rolling circle replication, mixing a secondary strand displacement primer and a tertiary strand displacement primer with the R-DNA and incubating to replicate the reiterated sequence DNA—where replication of the reiterated sequence DNA results in the formation of secondary reiterated sequence DNA and where replication of the secondary reiterated sequence DNA results in formation of tertiary reiterated sequence DNA (R-DNA-3).

Strand displacement replication can also be carried out sequentially. Following a first round of strand displacement replication, a tertiary strand displacement primer can be mixed with the R-DNA and R-DNA-2 and incubated to replicate the secondary reiterated sequence DNA, where replication of the secondary reiterated sequence DNA results in formation of tertiary reiterated sequence DNA (R-DNA-3). This round of strand displacement replication can be referred to as tertiary strand displacement replication. However, all rounds of strand displacement replication following rolling circle replication can also be referred to collectively as strand displacement replication.

A modified form of strand displacement replication results in amplification of R-DNA and is referred to as opposite strand amplification (OSA). OSA is the same as strand displacement replication except that a special form of rolling circle replication primer is used that prevents it from hybridizing to R-DNA-2. This can be accomplished in a number of ways. For example, the rolling circle replication primer can have an affinity tag coupled to its non-complementary portion allowing the rolling circle replication primer to be removed prior to strand displacement replication. Alternatively, remaining rolling circle replication primer can be crippled following initiation of rolling circle replication. One preferred form of rolling circle replication primer for use in OSA is designed to form a hairpin that contains a stem of perfectly base-paired nucleotides. The stem can contain 5 to 12 base pairs, most preferably 6 to 9 base pairs. Such a hairpin-forming rolling circle replication primer is a poor primer at lower temperature (less than 40° C.) because the hairpin structure prevents it from hybridizing to complementary sequences. The stem should involve a sufficient number of nucleotides in the complementary portion of the rolling circle replication primer to interfere with hybridization of the primer to the circular or circularized nucleic acid molecule. Generally, it is preferred that a stem involve 5 to 24 nucleotides, and most preferably 6 to 18 nucleotides, of the complementary portion of a rolling circle replication primer. A rolling circle replication primer where half of the stem involves nucleotides in the complementary portion of the rolling circle replication primer and the other half of the stem involves nucleotides in the non-complementary portion of the rolling circle replication primer is most preferred. Such an arrangement eliminates the need for self-complementary regions in the circular or circularized nucleic acid molecule when using a hairpin-forming rolling circle replication primer.

If an excess of tertiary reiterated sequence DNA is desired, the secondary strand displacement primer can be crippled in the same manner as is described above for the rolling circle replication primer (the rolling circle replication primer and tertiary strand displacement primer should not be crippled in this case). The reaction at the higher, permissive temperature should be carried out long enough to produce a reasonable amount of secondary reiterated sequence DNA to serve as a template for tertiary sequence DNA. When the temperature is shifted, the secondary strand displacement primer can no longer prime synthesis and the synthesis of tertiary reiterated sequence DNA soon outstrips the amount of secondary reiterated sequence DNA. Of course reiterated sequence DNA will continue to be produced by rolling circle replication throughout the reaction (since the rolling circle replication primer is not crippled).

When starting the rolling circle replication reaction, secondary strand displacement primer and rolling circle replication primer are added to the reaction mixture, and the solution is incubated briefly at a temperature sufficient to disrupt the hairpin structure of the rolling circle replication primer but to still allow hybridization to the primer complement portion of the circular or circularized nucleic acid molecule (typically greater than 50° C.). This incubation permits the rolling circle replication primer to hybridize to the primer complement portion of the circular or circularized nucleic acid molecule. The solution is then brought to the proper temperature for rolling circle replication, and the rolling circle DNA polymerase is added. As the rolling circle reaction proceeds, R-DNA is generated, and as the R-DNA grows in length, the secondary strand displacement primer rapidly initiates DNA synthesis with multiple strand displacement reactions on R-DNA. These reactions generate R-DNA-2, which is complementary to the R-DNA. While R-DNA-2 contains sequences complementary to the rolling circle replication primer, the primer is not able to hybridize nor prime efficiently at the reaction temperature due to its hairpin structure at this temperature. Thus, there is no further priming by the rolling circle replication primer and the only products generated are R-DNA and R-DNA-2. The reaction comes to a halt as rolling circle amplification stops and R-DNA becomes completely double-stranded. In the course of the reaction, an excess of single-stranded R-DNA-2 is generated.

Another form of rolling circle replication primer useful in OSA is a chimera of DNA and RNA. In this embodiment, the rolling circle primer has deoxyribonucleotides at its 3′ end and ribonucleotides in the remainder of the primer. It is preferred that the rolling circle replication primer have five or six deoxyribonucleotides at its 3′ end. By making part of the rolling circle replication primer with ribonucleotide, the primer can be selectively degraded by RNAseH when it is hybridized to DNA. Such hybrids form during OSA as R-DNA-2 is synthesized. The deoxyribonucleotides at the 3′ end allow the rolling circle DNA polymerase to initiate rolling circle replication. RNAseH can then be added to the OSA reaction to prevent priming of R-DNA-2 replication.

Unligated linear nucleic acid molecules may be removed prior to rolling circle replication to eliminate competition between unligated linear nucleic acid molecules and the secondary strand displacement primer for hybridization to R-DNA. Alternatively, the concentration of the secondary strand displacement primer can be made sufficiently high so that it out-competes unligated linear nucleic acid molecule for hybridization to R-DNA. This allows strand displacement replication to be performed without removal of unligated linear nucleic acid molecules.

As an optional step, mRNA may be produced directly by addition of an RNA polymerase that will recognize a primer in the circularized nucleic acid moelcule. In this way, an mRNA can be amplified without the necessity of cloning the mRNA and insertion into a cell.

Detection with a Circular Nucleic Acid

The present invention also includes detection of target nucleic acid molecules. A circular nucleic acid is used to detect such target nucleic acid molecules. The circular nucleic acid comprises at least two parts. The first part contains a sequence that will hybridize to the target nucleic acid molecule of interest. The second part contains a sequence that enables detection of the amplified circular.

In one aspect of the present invention, the circular nucleic acid molecules can be constructed from linear short oligo fragments with self-ligation. The linear short oligo fragments may contain special hairpin structures so that they can be self-ligated to form circular nucleic acid molecules. Such a method offers advantages compared to the use padlock probes and additional template. The ligation efficiency is much higher, and it avoids mis-ligation to form larger linear strand or larger circular nucleic acid molecules.

In another aspect of the present invention, a full-length circular cDNA nucleic acid molecule can be constructed from a commercially available full-length cDNA clone library. A gene specific clone will be selectively amplified with PCR and then circularized with self-ligation. The resulting full-length circular cDNA nucleic acid molecules can be used for gene specific detection and amplification without needing to use TaqMan or RT-PCR.

The invention is particularly directed to compositions and methods useful for the amplification of nucleic acid molecules by reverse transcriptase-rolling circle amplification (RT-RCA). Specifically, the invention provides compositions and methods for the amplification of nucleic acid molecules in a simplified RT-RCA procedure using combinations of reverse transcriptase and isothermal strand-displacement enzymes. The invention thus facilitates the rapid and efficient amplification of nucleic acid molecules and the detection and quantification of RNA molecules. The invention also is useful in the rapid production and amplification of cDNAs (single-stranded and double-stranded) which may be used for a variety of industrial, medical and forensic purposes.

In one embodiment, the circular nucleic acid molecules may optionally comprise additional defined sequences that may by used in later cloning, detection, amplification, or generation of RNA. Without limiting the generality of the foregoing, such defined sequences include restriction endonuclease sites, RNA polymerase promoter sites, polymerase termination sites, randomized sequences of short length such as a hexamer, a heptamer, an octamer, a nonamer, a decamer, an undecamer, or a dodecamer. The target nucleic acid molecule is hybridized to the circular nucleic acid. Once hybridized, rolling circle amplification can be initiated using the target nucleic acid molecule as a free 3′ end to initiate rolling circle amplification. This is in contrast to previous methods of detection using rolling circle amplification that rely upon ligation to form the circular nucleic acid. However, in certain embodiments, a linear strand may be used for detection that needs to be ligated for RCA to begin. This may be used to increase the sensitivity of detection. The present invention utilizes a nucleic acid that has already been circularized prior to addition to the sample and the target nucleic acid itself provides the free 3′ end. The above methods of generating a free 3′ end may be used to generate one or more free 3′ ends as desired. One of skill in the art will recognize that primers may be used to provide free 3′ ends as long as care is taken in the design of such primers that the primer will not hybridize to the circular nucleic acid and allow RCA directly. Thus, the primer may have a sequence at its 3′ end that is the same as the circular nucleic acid. Such primers will allow (n!) factorial amplification. It is preferred that the primer not hybridize to the target nucleic acid molecule. In one embodiment, the circular nucleic acid molecule further comprises a poly-A portion. This embodiment may be used in detection of an mRNA of interest. Once the target nucleic acid of interest has bound to the circular nucleic acid and rolling circle amplification has begun, the poly-A tails of the mRNA will bind to the nascent nucleic acid with the complementary poly-T portion. Thus, in such embodiment, (n!) factorial amplification may be achieved without addition of any primers.

The methods of the current invention may also be applied to detection of mutations. By careful selection of the method of generating free 3′ ends, reaction conditions may be selected whereby only mutant target nucleic acid molecules are amplified or only non-mutant target nucleic acid molecules are amplified. One example is targeted degradation of RNA by RNAseH using DNA hairpins. If the target nucleic acid molecule carries a single nucleotide polymorphism, the DNA at the hairpin will not anneal and RNAseH will not digest the RNA and generate a free 3′ end. Thus, RCA will not be initiated by mutant target nucleic acid molecules.

Detection of amplified product may be performed using any suitable technique for detection of nucleic acids. A few examples of detection methods are dyes that either directly or though an additional linked moiety interact with the nucleic acid by covalent linkage, intercalation, or some other form of binding. Radiolabels may also be used.

The detection methods of the present invention may also be used in multiplexed reactions, i.e., the simultaneous detection of two or more nucleic acids in a single sample. One of skill in the art may employ any suitable method for multiplexed detection of nucleic acids. In general, the products of the rolling circle amplification must be differentiable. This may be due to incorporation of different labels into the amplified products, different lengths of the products, or different sequences of the products. An example of a method of incorporation of different labels is to use different secondary primers with label attached. In designing circular nucleic acids for multiplexed detection, it is preferred that the regions complementary to the target nucleic acids be substantially different to limit non-specific priming of the rolling circle amplification reaction. Ideally, any circular nucleic acid should be designed to limit non-specific priming by non-target nucleic acids that may be in a mixture with the target nucleic acid.

Detecting Products

To aid in detection and quantitation of nucleic acids amplified using rolling circle amplification for cloning or detection of nucleic acids, detection labels can be directly incorporated into amplified nucleic acids, or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid or antibody probes are known to those of skill in the art. Examples of detection labels suitable for use in rolling circle amplification are radioactive isotopes, fluorescent molecules, phosphorescent molecules, enzymes, antibodies, nucleic acid binding proteins, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodo-le (DAPI), and the cyanine dyes Cy3, Cy3.5, Cy5, Cy5.5 and Cy7. Preferred fluorescent labels are fluorescein (5-carboxyfluorescein-N-hydroxysuccini-mide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 rim), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are preferred form of detection label since they can be directly incorporated into the products of rolling circle amplification during synthesis or affixed after synthesis. Examples of detection labels that can be incorporated into amplified nucleic acid products include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acids, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-(1,2,-dioxetane-3-2′-(5′-chloro)tricycle(3.3.-1.1.sup.3,7)decane)-4-yl)phenyl phosphate; Tropix, Inc.).

A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Molecules that combine two or more of these detection labels are also considered detection labels. Any of the known detection labels can be used with the disclosed probes, tags, and method to label and detect nucleic acid amplified using the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules that interact with amplified nucleic acid and to which one or more detection labels are coupled.

### EXAMPLES

The following examples are included to demonstrate various embodiments of the invention. It should be appreciated by those of skill in the art that the techniques disclosed in the examples are representative embodiments of the present invention, but the present invention is not limited to the examples presented herein. Those of skill in the art will, in light of the present disclosure, appreciate that many alternative embodiments to those disclosed herein exist and will yield similar results without departing from the spirit and scope of the invention.

Section I

Obtaining a full-length cDNA is one of the most important, and often one of the most difficult, tasks in characterizing genes. Traditional methods for cDNA library construction usually produce only partial cDNA fragments. To facilitate recovery of the rest of the coding sequence, an in vitro method for the rapid amplification of cDNA ends (RACE) was proposed in 1988. In spite of various modifications that have been developed, the current RACE technologies are complicated and inefficient. The present invention using RT-RCA technology provides a method that significantly simplifies the procedures to make full-length cDNA.

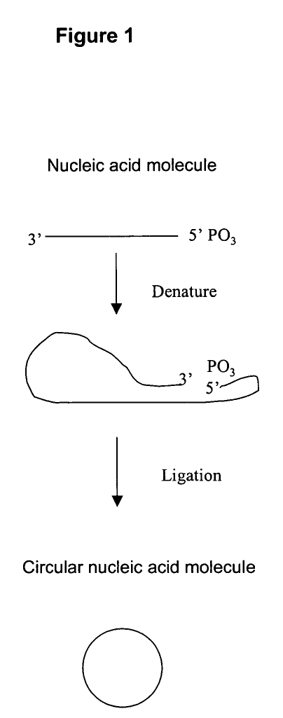

The flow chart that describes the RT-RCA technology is shown in FIG. 1. The preferred protocol is described below.

### Example 1

### Synthesis of the cDNA with Complementary Ends

MMLV reverse transcriptase (RT) has the ability to add cytosine residues to the 3′ end of newly synthesized cDNAs upon reaching 5′-end of the mRNA template. Usually 2-4 cytosine residues are added, depending on the reaction conditions.

mRNA is purified using standard methods that prevent RNA degradation. Small amounts of mRNA, as low as picrogram amounts, are used as the target nucleic acid molecule. A first strand synthesis primer containing poly(dT) and a T7 transcriptional promoter at its 5′ end, primer 1, and MMLV reverse-transcriptase enzyme are added to the mRNA sample. The poly(dT) sequence of the first strand synthesis primer anneals to the poly(A) tail of mRNA, serving as a primer for reverse-transcriptase to synthesize first strand cDNA. Simultaneously, primer 2 anneals to primer 1. At the 3′ end of the first strand cDNA, reverse-transcriptase adds a few cytosine residues. The 5′ end of first strand cDNA has the T7 promoter followed by a poly(T) stretch, as this sequence was used as the primer for the first strand synthesis. The T7 promoter is oriented such that once the molecule is circularized the promoter will direct transcription of a copy of the original mRNA.

Primer 1: 5′-d(T7 promoter sequence)+d(T)15-3′

Primer 2: 5′-d(T7 promoter sequence complement)+d(G)₄-3′

10 pmol of cDNA synthesis primers are annealed to 1 μg of human placenta poly(A)⁺ RNA (Clontech), in a volume of 5 μL of deionized water, by heating the mixture for 2 minutes at 70° C., followed by cooling on ice for 2 minutes. First-strand cDNA synthesis is then initiated by mixing the annealed primer-RNA complexes with 200 units of M-MLV Rnase H-reverse transcriptase (superscript II reverse transcriptase, Life Technology) in a final volume of 10 μl containing 50 mM Tris-HCl (pH 8.3 at 22° C.); 75 mM KCl; 6 mM MgCl₂; 1 mM DTT; and 1 mM each of dATP, dGTP, dCTP, and dTTP.

To the above reaction solution, 1 U of RNAse H is added and incubated for 1.5 hours. The resulting solution is purified with Qiagen kit and then detected with UV absorbance to measure the amount of cDNA with Nanodrop instruments. Optical density indicates about 140 ng of cDNA is obtained. The resultant nucleic acid will have a 3′ overhang of d(C) on one end and a 3′ overhang of d(G) on the other end. The overhangs may anneal and allow template switching thus generating a circular molecule. Ligase may be added to link the ends of the molecules.

### Example 2

### Synthesis of the cDNA with LoxP Recombination Sites

A LoxP recombination site may be added by oligo switch technology. An oligonucleotide with oligo(G) or oligo(rG) sequences at its 3′ most end is included in the first strand cDNA synthesis medium. Its terminal 3-4 G residues will base pair with the 2-4 C residues of the newly synthesized cDNA, thus serving as a new template for the RT (template switch). The RT then switches the template and replicates the sequence of the oligo(G) oligonucelotide, thus including the complementary CapFinder oligonucleotide sequence at the 3′ end of the newly synthesized cDNA.

Primer 3: 5′-d(LoxP sequence)+d(T)15-3′

Primer 4: (sequence for oligo switch) 5′-d(LoxP sequence)ᵣ(GGGp)-3′

10 pmol of cDNA synthesis primer 3 are annealed to 1 μg of human placenta poly(A)⁺ RNA (Clontech), in a volume of 5 μl of deionized water, by heating the mixture for 2 minutes at 70° C., followed by cooling on ice for 2 minutes. First-strand cDNA synthesis is then initiated by mixing the annealed primer-RNA complex with 200 units of M-MLV Rnase H-reverse transcriptase (superScript II reverse transcriptase, Life Technology) in a final volume of 10 μl containing 50 mM Tris-HCl (pH 8.3 at 22° C.); 75 mM KCl; 6 mM MgCl₂; 1 mM DTT; and 1 mM each of dATP, dGTP, dCTP, and dTTP. The first-strand cDNA synthesis-template switching reaction is incubated at 42° C. for 1.5 hours in an air incubator, and then cooled on ice.

To the above reaction solution, 1 U of RNAse H is added and incubated for 1.5 hours. The resulting solution is purified with Qiagen kit and then detected with absorbance to measure the amount of cDNA with Nanodrop instruments. The OD indicated about 160 ng of cDNA is obtained.

### Example 3

An alternative method is to use terminal transferase enzyme to add homologous sequences to the 3′ end of the first strand cDNA.

The synthesized first strand cDNA is purified with Qiagen kit. Then the first strand 0.5 ug cDNA is mixed with 0.5 uM dCTP, 1× Reaction buffer of Terminal Transferase and 1 unit of Terminal transferase (Finnzymes) at 37 degree for 1.5 hours. The resulting solution is purified with Qiagen kit and detected with Bioanalyer (Agilent). It is finally quantified with Nanodrop absorbance indicating 0.45 ug of cDNA.

### Example 4

### Synthesis of the Circular Molecule

The first strand cDNA can be circularized with ligation by using a short oligonucleotides as a bridge. After the first strand cDNA is synthesized, an oligonucleotide with sequences complementary to the sequences at both 3′ end and 5′ end of the newly synthesized cDNA is incubated with T4 DNA ligase in the incubation medium. The resulting cDNA will be circularized.

Primer 5: 5′-d(Complementary sequence of T7)+dGdGdG-3′

Incubate the first strand cDNA with T4 DNA ligase (Promega) in 1×T4 DNA ligation buffer (Promega), 0.5 mM ATP and primer 5 at room temperature overnight. Then 0.5 U Exonuclease V (Amersham) and 0.5 mM ATP are added to the above solution for another 1.5 hours. All the linear strand DNA is digested. The resulting circular cDNA is purified with Qiagen kit and measured with absorbance. 0.5 ug circular cDNA is produced.

### Example 5

### Circularization by Randomer Hybridization

The present invention may be used to amplify double stranded target nucleic acid molecules. The following example illustrates a method of amplifying an entire target nucleic acid without reference to the sequence of the target. As such, the method could be adapted for amplification of entire genomes or other large samples of double stranded nucleic acid molecules.

Total genomic DNA is digested with Pml I in 10 mM Bis Tris Propane-HCl, 10 mM MgCl₂, 1 mM dithiothreitol (pH 7.0 @ 25° C.), 100 μg/ml BSA, 100 μM dNTPs by incubating at 37° C. for one hour.

T4 DNA polymerase is added to the mix with excess of a linker oligonucleotide containing in the 5′ half a LoxP site for CreI dependent recombination and a random hexamer sequence at the 3′ end. The reaction is incubated overnight at 37° C. The resulting products will be genomic fragments with a LoxP site at either end of the nucleic acid.

Crel is then added to the sample and incubated at 37° C. for one hour. The resulting products are circularized fragments of the entire genome suitable for rolling circle amplification.

### Example 6

### Circularization by Recombination

First strand cDNA can be circularized with Cre-recombinase. First strand cDNA is synthesized with loxP sites at both 3′ end and 5′ end as described in Example 2. Incubation the first strand cDNA with CreI recominbinase in an incubation medium will circularize the first strand cDNA.

First strand cDNA synthesized with LoxP sequences at both the 3′ end and the 5′ end is incubated with cre-recombinase at 37° C. for 4 hours. The recombinase is deactivated by increasing the temperature to 75° C. for 30 min. Then 0.5 U Exonuclease V (Amersham) and 0.5 mM ATP are added to the above solution and incubated for 1.5 hours. The circularized cDNA is purified with Qiagen kit and measured with absorbance (0.6 ug).

### Example 7

### Amplification of the Circular cDNAs

Circularized cDNA can be amplified with rolling circle amplification by performing a limited RNaseH digest of an mRNA:first strand cDNA complex. The resulting nicked mRNA can be used as primers to perform RCA amplification by virtue of the free 3′ ends. This method will yield free 3′ ends in only one strand as is required for RCA.

Circularized cDNA with RNA:DNA duplex is incubated with 0.5 U RNase H at 37° C. for 30 minutes. Then 4 μl of the above reaction is incubated in a volume of 35 μl containing 20 mM Tris.HCl (pH=8.8), 10 mM KCl, 2.7 mM MgSO4, 5% v/v DMSO, 0.1% Triton X-100, 400 μM dATP, dGTP, dCTP, DTTP and 900 nM of Primer 5 (AGGCCTGCATTATTCC (SEQ ID NO:1), a primer for exponential amplification). Phage T4 gene-32 protein (Amersham) is present at a concentration of 38 ng/uL, (approximately 1085 nM). After combining all the materials at RT, the reactions are placed on ice, Vent (exo_) DNA polymerase (New England Biolabs) is added at a final concentration of 0.32 units/ul, and the reactions are incubated at 75 degree for 3 min, then at 65.5° C. for 90 mins. The resulting mixture is run on a gel. The rolling circle products are observed at the top of the gel after staining, due to not having entered the gel.

### Example 8

### Amplification of cDNAs with Random Priming

Circularized cDNA can be amplified with rolling circle amplification by using random hexamer as primers. The random hexamers will only amplify circular nucleic acid molecules. RNA digestion or heat denaturation is used to disassociate the mRNA from the first strand circular cDNA. Heat denaturation may also be used to dissociate dsDNA in preparation for amplification. The ssDNA may then be isolated for use as a reagent in other biological applications. To the ssDNA, random hexamer can be added along with a strand displacement polymerase such as Phage 29, vent and BST. The mixture would be incubated at the appropriate temperature for the strand displacement polymerase for the desired period of time.

4 ul of single strand circularized DNA is incubated in a volume of 35 ul containing 20 mM Tris.HCl (pH=8.8), 10 mM KCl, 2.7 mM MgSO4, 5% v/v DMSO, 0.1% Triton X-100, 400 uM dATP, dGTP, dCTP, DTTP and 900 nM of the Random Hexamer (Amersham). Phage T4 gene-32 protein (Amersham) is present at a concentration of 38 ng/ul, (approximately 1085 nM). After combining all these materials at RT, the reactions are placed on ice. Vent (exo-) DNA polymerase (New England Biolabs) is added to a final concentration of 0.32 units/ul, and the reactions are incubated at 75° C. for 3 minutes, then at 65° C. for 90 minutes. The resulting mixture is run on a gel. The rolling circle products are observed at the top of the gel after staining, due to not having entered the gel.

### Example 9

### In Vitro RNA Transcription

In vitro RNA transcription may be conducted using any of the above circular cDNA as a template. With the addition of T7 polymerase and rNTPs, T7 polymerase will transcribe either the sense or antisense strand of the cDNA depending upon the selected orientation of the T7 promoter.

The resulting double strand RCA products are transcribed with T7 RNA polymerase. 3 ng of cDNA is transcribed in each reaction. Reactions conditions are: 40 mM Tris pH 7.5, 6 mM MgCl₂, 10 mM NaCl, 2 mM spermidine, 10 mM DTT, 500 μM each ATP, GTP, and UTP-cy3, 12.5 μM CTP, 10 units Rnase block, and 80 units T7 RNA polymerase in a volume of 20 μl. Reactions are incubated at 37° C. for 2 hour. The resulting mixture is purified with a Qiagen kit. The synthesized dye labeled aRNA is eluted with ethanol and measured with Nanodrop.

Section II

RT-PCR (reverse transcription-polymerase chain reaction) is a highly sensitive technique for mRNA detection and quantization. The technique consists of two parts: synthesis of cDNA from RNA by reverse transcription (RT) and amplification of a specific cDNA by polymerase chain reaction (PCR). The present invention offers a simpler method of mRNA amplification and detection using rolling circle amplification.

A gene specific circular nucleic acid molecule contains at least two segments. The first segment contains a sequence complementary to the target nucleic acid molecule. The second segment contains zip code sequences for detection. The following sequence is used as an example to amplify and detect house keeping gene GAPDH.

The gene specific sequence for GAPDH is 5′-AGGTTTTTTCTAGACG-3′ (SEQ ID NO:2) (16 mer). The zip code sequence for detection is 5′-CATCGTCCCTTTCGATGGGATCM-3′ (SEQ ID NO:3) (24 mer). The full-length sequence

(SEQ ID NO:4)

5′- TTCTAGACGCATCGTCCCTTTCGATGGGATCAAAGGTTT -3′.

### Example 10

### Circularization of the Detection Circle

The linear full length sequence is obtained with the 5′ end phosphorylated (Integrated DNA Technologies, Coralville Iowa). This linear sequence is circularized by using the following template sequence: 5′ TCCAAAAAGATCTGC-3′ (SEQ ID NO:5).

The circularization reaction contains 50 μM circle precursor, 50 μM template, 100 mM NiCl₂, 200 mM imidazoleHCl (pH=7.0), and 125 mM BRCN, and the reaction is allowed to proceed 10 h at 23° C. After dialysis and lyophilization the product is purified by preparative denaturing 20% polyacrylamide gel electrophoresis, and the product band is isolated by excision, crushing, and eluting into 0.2 M NaCl. The salts are removed by dialysis against distilled deionized water, and the DNA is quantitated by absorbance at 260 nm, using the nearest neighbor method to calculate molar extinction coefficients.

### Example 11

### mRNA Amplification and Detection

Total RNA is obtained from Clontech. The total RNA is pre-processed with a ribozyme that cleaves the GAPDH mRNA at the 3′ end of the sequence CGUCUAGAAAAACCU (SEQ ID NO:6). 0.5 μg of processed total RNA is mixed with the circular oligonucleotide prepared in Example 9 in 20 mM Tris. HCl (pH=8.8), 10 mM KCl, 2.7 mM MgSO4, 5% v/v DMSO, 0.1% Triton X-100, 400 μM dATP, dGTP, dCTP, dTTP. The mixture is heated to 75° C. for 5 minutes. Then the mixture is cooled to room temperature slowly. To the above reaction are added 1 U phage 29 (Amersham), 1 U RNaseH and Phage T4 gene-32 protein (Amersham) with a concentration of 38 ng/ul. The resulting mixture is incubated at 37° C. for 4 hours. Then the reaction mixture is incubated at 95° C. for 10 min to deactivate all the enzymes. The double strand cDNA is precipitated out with phenol chloroform. The precipitated DNA is run on a gel and stained. Gel staining shows the long double strand cDNA located at the top of the wells. Detection can be performed by any of the methods available to one of skill in the art. To enhance amplification, the primer 5′-GTCCCTTTCGATGGG (SEQ ID NO:7) may be added to the above reaction. Addition of this primer will result in (n!) factorial amplification.

### Example 12

### Multiplexed Detection Reaction

For detection of multiple genes in a single tube, the same reaction as described in Example 11 can be carried out by combining multiple gene specific or mRNA specific circular templates in the same reaction.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

1-53. (canceled)

54. A method of amplifying a polynucleotide, comprising:
a) forming a linear polynucleotide having 3′ and 5′ hairpins;
b) ligating 3′ and 5′ ends of the linear target to form a circularized polynucleotide; and
c) amplifying the circularized polynucleotide by rolling circle amplification.

55. The method of claim 54, further comprising prior to the forming step:
a) hybridizing a first hairpin to a 3′ portion of a target nucleic acid; then
b) polymerizing copy DNA from the 3′ end of the first hairpin using the target as a template; and
c) affixing a second hairpin to a 5′ portion of the target or a 3′ portion of the copy DNA to form the linear polynucleotide.

56. The method of claim 55, wherein the affixing step is effected by self-priming.

57. The method of claim 56, wherein the affixing step is effected by self-priming using a template switching oligonucleotide to extend the target nucleic acid.

58. The method of claim 57, wherein the template switching oligonucleotide comprises a restriction site, the first hairpin is formed by cutting double-strand DNA of target DNA and template switch oligonucleotide at the restriction site to form a cut end, and ligating to the cut end a corresponding cut end of a hairpin.

59. A method according to claim 54 for making circular copy DNA by:
a) hybridizing a first primer to a 3′ portion of a template region of a target strand;
b) polymerizing from the primer a first copy DNA of the template region;
c) displacing from the template region the first copy DNA;
d) forming a hairpin second primer at a 3′ portion of the first copy DNA;
e) polymerizing from the hairpin primer a second copy DNA of a portion of the first copy DNA; and
f) ligating the 5′ end of the first copy DNA with the 3′ end of the second copy DNA to form a circular copy DNA.

60. A method according to claim 59, wherein the displacing step is effected with nuclease, base or strand displacement.

61. A method of amplifying a DNA comprising:
a) polymerizing a copy DNA of a template region of a DNA target to form a double-stranded DNA having first and second hairpins at first and second ends, respectively;
b) ligating the double-stranded DNA in a single molecular reaction to form a circularized DNA; and
c) amplifying the circularized DNA by rolling circle amplification.

62. The method of claim 61, wherein the target is transcribed from an mRNA

63. The method of claim 61, wherein the target is transcribed from an mRNA and the first hairpin is formed by self-priming of the target.

64. The method of claim 61, wherein the target is transcribed from an mRNA with at least one template switching hairpin oligonucleotide, the first hairpin is formed by covalently attaching said template switching hairpin oligonucleotide to the 3′ end of the target, optionally with the template switching hairpin oligonucleotide serving as a template for extension of the 3′ end of the target.

65. The method of claim 61, wherein the target is transcribed from an mRNA, the second hairpin is formed by initiating polymerization of the target with a hairpin primer, wherein the hairpin is formed before or after polymerization of the target.

66. The method of claim 61, wherein the target is transcribed from an mRNA using a primer comprising a restriction site to initiate polymerization of the target, the second hairpin is formed by cutting the double-stranded DNA at the restriction site to form a cut end, and ligating to the cut end a corresponding cut end of a hairpin.

67. The method of claim 61, wherein the target is genomic DNA, wherein the first and second hairpins are formed by ligating first and second ends of the genomic DNA to corresponding cut ends of corresponding hairpins.

68. The method of claim 61, wherein the target is a single stranded DNA copied from a genomic DNA or cDNA library or transcribed from an mRNA, the first and second hairpins are formed by self-hybridization of 3′ and 5′ ends of the target DNA at adjacent positions, and the ligating step comprises covalently linking the 3′ and 5′ ends to form the circularized DNA.

69. The method of claim 61, wherein the target is transcribed from an mRNA with a template switching oligonucleotide comprising at its 3′end at least one nucleotide which basepairs with a nucleotide at the 3′ end of the target strand of an RNA-DNA intermediate comprising the mRNA and the target, wherein the template switching oligonucleotide serves as a template for extension of the target, and has a pre-selected arbitrary nucleotide sequence at its 5′ end, and extending the 3′ end of the target to produce a DNA that is complementary to the mRNA molecule and the template switching oligonucleotide.

70. A method of amplifying a linear nucleic acid target, comprising steps:
a) affixing a first oligonucleotide linker comprising a hairpin to one end of the target;
b) forming a hairpin at the other end of the target;
b) circularizing the target;
c) generating a free 3′ end on the target; and
d) amplifying the target from the free 3′ end by rolling circle amplification.

71. The method of claim 70 wherein the circularizing step is performed by a method selected from the group consisting of annealing complementary ends followed by ligation, and self-priming followed by ligation.

72. The method of claim 70 wherein the target nucleic acid molecule is a first strand cDNA.

73. The method of claim 70 wherein the first linker is affixed by hybridization and ligation, hybridization followed by polymerase extension, enzymatic reaction, chemical reaction, and photo-reaction.

74. The method of claim 70 further comprising affixing a second oligonucleotide linker prior to circularization.

75. The method of claim 74 wherein the second linker is affixed by a method selected from the group consisting of ligation, hybridization and ligation, hybridization followed by polymerase extension, self-priming and ligation, enzymatic reaction, chemical reaction, and photo-reaction.

76. The method of claim 70 wherein the first linker is affixed by randomer hybridization and extension from nucleic acid breath.

77. The method of claim 70 wherein the generating step comprises adding one or more primers comprising the free 3′ end.

78. The method of claim 70 wherein the generating step comprises nicking a strand of target, wherein the target is double-stranded.

79. The method of claim 77 wherein the one or more primers further comprise random sequences at their 3′ ends.

80. The method of claim 77 wherein the one or more primers further comprise promoter sequences.

81. The method of claim 77 wherein the one or more primers are RNA:DNA chimeras.

82. The method of claim 70, wherein the circularized target comprises a sequence for in vitro or vivo protein expression.


SEQUENCE LISTING (7 sequences)

SEQ ID NO: 1 - DNA, 16 bp, Artificial
  Synthetic primer/probe
aggcctgcattattcc

SEQ ID NO: 2 - DNA, 15 bp, Artificial
  Synthetic primer/probe
aggtttttctagacg

SEQ ID NO: 3 - DNA, 24 bp, Artificial
  Synthetic primer/probe
catcgtccctttcgatgggatcaa

SEQ ID NO: 4 - DNA, 39 bp, Artificial
  Synthetic primer/probe
ttctagacgcatcgtccctttcgatgggatcaaaggttt

SEQ ID NO: 5 - DNA, 15 bp, Artificial
  Synthetic primer/probe
tccaaaaagatctgc

SEQ ID NO: 6 - RNA, 15 nt, Artificial
  mRNA target sequence
cgucuagaaaaaccu

SEQ ID NO: 7 - DNA, 15 bp, Artificial
  Synthetic primer/probe
gtccctttcgatggg